(19) United States
(12) Patent Application Publication
(10) Pub. No.: US 2025/0275760 A1
(43) Pub. Date: Sep. 4, 2025

(54) Punch Biopsy Apparatus with Sample Coring, Cutting, and Sample Retrieval Mechanism

(71) Applicants: Eduardo A. Michelen-Gomez, San Juan, PR (US); Angel R. Gomez-Cruz, San Juan, PR (US)

(72) Inventors: Eduardo A. Michelen-Gomez, San Juan, PR (US); Angel R. Gomez-Cruz, San Juan, PR (US)

(21) Appl. No.: 18/593,900

(22) Filed: Mar. 2, 2024

(51) Int. Cl. A61B 10/02

(52) U.S. Cl. CPC A61B 10/0266; A61B 2010/0208

(57) ABSTRACT

A punch biopsy device with a sample coring and cutting mechanism actioned by a cutter actuator which is pushed when the sample is cored from the patient, engaging the cutting function and separating the sample from the patient. The cutter actuator is then disengaged to retrieve the sample making it easier for retrieval and less likely that the sample will be damaged from the process of retrieval, as is more likely to happen from the use of the common punch biopsy device found in most hospitals and dermatology offices.

### RELATED APPLICATIONS

N/A

### BACKGROUND OF THE DISCLOSURE

### Technical Field

The present disclosure relates generally to the field of extracting tissue samples from solid bodies, and more specifically to surgical instruments, such as a punch biopsy device for extracting a biopsy or sample of tissue.

### Discussion of the Background

Punch biopsy devices are mainly used in hospitals and dermatologists' offices. Said punch biopsy devices are used to core a sample of the patient's skin, epidermal, dermal and sub-dermal layers, to obtain said cored sample and use it for different tests to determine which skin condition, if any, the patient may have. The most common punch biopsy devices do include a sample coring device. However, the most used and most accessible punch biopsy devices don't have a cutting mechanism to cut the sample free. The known punch biopsy devices may be twirled and fidgeted side to side to free the sample within the device. The device may also be angled to cut and retrieve the sample. These techniques may work in cutting the sample free but are very likely to damage said sample. Other techniques include the use of other instruments to cut the sample free and retrieve it, such as the utilization of a blood drawing needle or the use of tweezers. While these methods may succeed in cutting the sample free from the patient and retrieving it, they are also likely to damage the sample as well as use and soil single-use sterile instruments and waste more money.

The main disadvantage of damaging the sample is the need to take another sample, put the patient through more pain and discomfort, and have the possibility of ruining that sample as well, creating a cycle of pain until a usable sample is obtained, or the use of a damaged sample to run the necessary tests and possibly yielding erroneous results.

US patent application US20220354470A1 proposes a punch biopsy device with a cylindrical body with a griping portion at the top of the body and a cylindrical needle attached at the bottom of the body used to pierce the skin of the patient and core the sample, an independent rotating blade to cut the base of the sample, a plunger to secure the sample for extraction from the patient, and a compartment for expulsion of skin glue to close the wound once the sample is extracted. This device's advantages are that it can cut the sample at the base, free it, and retrieve it from the patient. Another advantage is the skin glue application to close the wound without sutures. However, one of the main disadvantages of this device is that it isn't readily available on the market at the moment of filing this application, making the cost impossible to predict. The other disadvantage is that this device is complex in that it contains multiple moving and cutting parts between the coring aspect of the device and the base cutting aspect of the device as well as a glue dispenser that, if placed incorrectly during the coring process, can damage the sample with one of the coring or cutting blades or contaminate the sample with glue, causing the necessity to perform another punch biopsy. The cutting mechanism is different to the one on the punch biopsy device proposed in this application. It is prone to more human error given it has multiple moving parts.

US patent application publication US2015/0057572 demonstrates a punch biopsy device that comprises an elongated hollow cylinder with a distal end having a preloaded exposed cylindrical cutter which rotates during the incision procedure and is then retracted into the body of the elongated hollow cylinder while the cylindrical cutter assists with the grasping and removing of the tissue sample and; wherein said cylindrical cutter is coupled to a suture-less fastening mechanism. Said cuter is comprised of a plurality of blades, a middle cuter body, a tapered gap, a cuter base and fixing means. The cutting mechanism is engaged when the instrument pierces the skin and is twisted. A big advantage to this device is the suture-less fastening mechanism that closes the wound when retracting the cutters. This is also one of its biggest disadvantages because if applied wrongly, the suture-less wound closure might have to be removed and a suture applied, causing the patient more stress and, possibly, pain. Other disadvantages include the possibility of the sample becoming stuck in the instrument. Though this instrument may achieve the cutting of the base of the cored sample, the overall complicated design and mechanism will likely increase the price as well as the possibility of inadequate use and possible damage to the sample as well as the patient's skin.

U.S. Pat. No. 8,936,557B2 expresses a punch biopsy device with a cylindrical body containing a gripping portion at the top of the body and a scoop shaped blade attached at the bottom of the body and two arms attached to the body just above the blade to help guide the blade to perform a punch biopsy or hair follicle retrieval. Advantages to this device are that it properly cores the sample and cuts the base once properly inserted and twisted 360°, making retrieving the sample possible without the use of additional instruments. A disadvantage to this device is that even though human error in the coring aspect of the device is limited because of the arms that help guide the procedure, the depth of the necessary coring may render the arms useless or even a disturbance in certain situations where the necessary depth of the core doesn't meet the standards of the arms. Also, the coring mechanism needs to be inserted at an angle, increasing the likelihood of a mistake caused by human error. Another disadvantage is the fact that the depth of the insertion of the scoop-shaped blade directly affects the diameter of the sample and the wound.

US patent application US20090018467A1 proposes a punch biopsy apparatus for removing all or a portion of a suspect dermal growth. Said punch biopsy apparatus has a hollow cylinder body, a coring blade at the base of the hollow cylinder body, at least one scooping blade pivotally secured by an axle and a pair of pivot seats within the cylinder body. where each scooping blade is semicircular, a longitudinally-moveable plunger secured within the hollow cylinder, where the plunger has at least one plunger leg for contacting each scooping blade, and each plunger leg transfers longitudinal plunger movement to its respective scooping blade, thereby causing rotational scooping-blade movement. The advantage of this instrument is its ability to cut the base of the cored sample. A disadvantage is the complexity of the design. This will likely increase the price as well as the possibility of inadequate use and the damage of samples. Another disadvantage is that the deeper the biopsy, the greater the surface area of the sample and the wound.

US patent application US20150126903A1 expresses a punch biopsy tool that includes a punch cutting element which itself includes an inner surface, an outer surface, and a cutting edge along a bottom edge, and which defines an aperture between the inner surface and the outer surface. The punch biopsy tool also includes a flexible cutting element which is disposed along the inner surface of the punch cutting element and is configured to be pulled through the aperture. This can be used to cut the base of the cored sample. An advantage to this device is that it can cut the base of the cored sample, effectively separating the sample from its base and making the retrieval of the sample possible without the use of other instruments. A disadvantage is the complexity of the design. This will likely increase the price as well as the possibility of inadequate use and the damage of samples. Another disadvantage is the complexity of the cutting mechanism, which increases the necessary steps to engage it, prolonging the duration of the process and causing the patient possible discomfort.

Further, the commonly used punch biopsy device is a plastic cylindrical body with a sharp steel coring cylinder. The coring cylinder is used to pierce the skin and core the sample; however, it doesn't cut the sample at the base and free it from the patient. Leaving the performer of the biopsy to decide whether to angle the device to attempt to cut the sample free with the steel coring aspect of the device or to remove the device and attempt to use tweezers and a blood drawing needle or scissors or other cutting means to separate the sample from the patient. These techniques pose a disadvantage because they may ruin the sample distorting the biopsy's morphology soil other sterile, single-use instruments, causing hospitals or medical offices and service providers to waste more money on more instruments. If able to free the sample using the common punch biopsy device, the sample may also get stuck in the device.

It is, therefore, desirable to create a device capable of performing a punch biopsy by coring a sample of the patient's skin and containing a mechanism capable of cutting said sample free from the patient without damaging the sample or causing the patient any more pain or discomfort than necessary. It is also desirable that said device also contains a method or mechanism for retrieving said sample from the patient. Therefore, the present disclosure is directed to overcome the problems or disadvantages associated with the prior art.

### SUMMARY OF THE DISCLOSURE

In accordance with the principles of the current disclosure, the present exemplary embodiment is directed toward a punch biopsy device configured to core a sample of the patient's epidermal, dermal, and sub-dermal layers of skin, cutting said cored sample free from the patient and retrieve it without damaging the sample. The present invention comprises a punch biopsy device with at least two-piece interconnected bodies, comprising a large main piece and one smaller secondary piece, wherein the secondary piece comprises a first sharp semi-cylindrical steel coring structure attached to the secondary piece distal end. Further the large main piece comprises a second sharp semi-cylindrical steel coring structure attached to the main piece distal end. The large main piece further comprises a hollow space with a hole at the bottom, wherein the hole may include several shapes including but not limited to a circular-like hole on the large main piece. The smaller secondary piece, or cutter actuator is attached to the top large main piece and is fed through the hole of said large main piece. A portion of the cutter actuator extends from the large main piece for acting as a button for engaging the core cutting and a retrieving mechanism for sample tissue when it is depressed.

The present disclosure comprises a two-piece body consisting of a long main piece with a hollow space including a hole, wherein the hollow space is defined by parallel up right small wings or walls extending from the main piece near a distal end of the main piece, wherein each wall comprises a hole. A shorter secondary piece attached to said longer main piece, wherein the shorter secondary piece comprises cylindrical pegs or a pivot element attached to said secondary piece that are fed through the holes in the walls of the long main piece keeping said secondary piece in place but allowing a rotational motion creating a opening and closing movement. Further the long cylindrical peg of the secondary body is used to attach the secondary piece to the main piece. The secondary piece further comprises a longer peg or in the alternative a pivot element, being fed through the circular hole of the main piece and functioning as a pivoting element to engage the shorter secondary piece with the long main piece allowing the rotational movement of the secondary piece while assisting the cutting function.

Another aspect of the present disclosure is that two cutting plates, each cutting plate with a sharp curved end defining a cylindrical shape, are attached to the distal ends of the two piece body, one on the distal end of the main piece and the other one on the distal end of the secondary piece, in such a way that when the actuator button for the cutting function isn't engaged, the steel plates are parallel to each other but when the button is engaged, these two steel plates are brought closer to each other achieving the tissue retrieving action by the cutting mechanism.

Another aspect of the present disclosure is that said cutting steel plates allow the instrument to pierce the skin of the patient and core a sample of patient's epidermal, dermal and sub-dermal layers of skin, depending on the depth desired by the doctor practicing the punch biopsy.

Another aspect of the present disclosure is that once the desired coring depth of the sample is achieved, the button may be pressed to engage the cutting mechanism and retrieve said sample from patient setting it free from the patient.

Another aspect of the present disclosure is that once the sample is cut, the sample can be retrieved by maintaining the cutting mechanism engaged, pulling the instrument out of the patient, and, once out releasing the sample, for example by pushing on the side of the secondary piece opposite to the button, disengaging the cutting mechanism, bringing the two steel plates back to the parallel position and releasing the sample.

The present disclosure may address one or more of the problems and deficiencies of the prior art discussed above. However, it is contemplated that the disclosure may prove useful in addressing other problems and deficiencies in several technical areas. Therefore, the claimed invention should not necessarily be construed as limited to addressing any of the particular problems or deficiencies discussed herein.

Therefore, those skilled in the art will appreciate that the conception, upon which this disclosure is based, may readily be utilized as a basis for the designing of other structures, systems, methods and media for carrying out several purposes of the disclosed subject matter. It is important, therefore, that the claims be regarded as including such equivalent construction insofar as they do not depart from the scope of the present subject matter.

### BRIEF DESCRIPTION OF THE DRAWINGS

Various features and advantages of the disclosed subject matter can be more fully appreciated with reference to the following detailed description in connection with the following drawings, in which like reference numerals identify like elements.

FIG. 1 illustrates an exemplary isometric view of the punch biopsy device in accordance with the disclosure.

FIG. 2 illustrates an exemplary isometric exploded view of the punch biopsy device in accordance with the disclosure.

FIG. 3 illustrates an exemplary front view of the punch biopsy device in accordance with the disclosure.

FIG. 4 illustrates an exemplary side view of the punch biopsy device in accordance with the disclosure.

FIG. 5 illustrates an exemplary top view of the punch biopsy device in accordance with the disclosure.

FIG. 6 illustrates an exemplary isometric back view of the punch biopsy device in accordance with the disclosure.

FIG. 7 illustrates an exemplary top view of the main body in accordance with the disclosure.

FIG. 8 illustrates an exemplary front view of the second embodiment of the punch biopsy device in accordance with the disclosure.

FIG. 9 illustrates an exemplary upper cutter isometric view of the second embodiment of the punch biopsy device in accordance with the disclosure.

FIG. 10 illustrates an exemplary isometric back view of the punch biopsy device in accordance with the disclosure.

FIG. 11 illustrates an exemplary top view of the main body in accordance with the disclosure.

FIG. 12 illustrates an exemplary isometric back view of the punch biopsy device in accordance with the disclosure.

FIG. 13 illustrates an exemplary top view of the main body in accordance with the disclosure.

FIG. 14 illustrates an exemplary flowchart of the method of using the punch biopsy device in accordance with the disclosure.

### DETAILED DESCRIPTION

In the following detailed descriptions of one or more preferred embodiments are provided herein. Numerous specific details are set forth regarding the system and method of the disclosed subject matter and the environment in which such systems and methods may operate to provide a thorough understanding of the disclosed subject matter. It will be apparent to one skilled in the art, however, that the disclosed subject matter may be practiced without such specific details, and that certain features, which are well known in the art, are not described in detail in order to avoid complications of the disclosed subject matter. However, it is to be understood that the present disclosure may be embodied in various forms. Therefore, specific details disclosed herein are not to be interpreted as limiting, but rather as a basis for the claims and as a representative basis for teaching one skilled in the art to employ the present disclosure in any appropriate system, structure, or manner that are within the scope of the disclosed subject matter.

In the present disclosure, the device could be manufactured using different types of materials. For example, the elongated body of the device can be made from plastic, carbon fiber, fiberglass, metal, or any other durable material that can be sterilized and in doing so is safe to wield near an open wound. Further, the cutter potion, the cutter actuator, the upper cutter extended end and the pivot element could be manufactured using different methods, including but not limited to, a single material similar to the main body or a different material including but not limited to the materials for the main body. The selection of material depends on several factors, including but not limited to, cost, area of the body wherein the cutter is going to be inserted, precision of the wound, safety, patient allergies and others. The selected material should allow the cutter to be inserted in a patient and the elongated main body should be able to withstand the pressure and/or force applied by the user for cutting without breaking or getting damaged during the procedure, surgery or the alike.

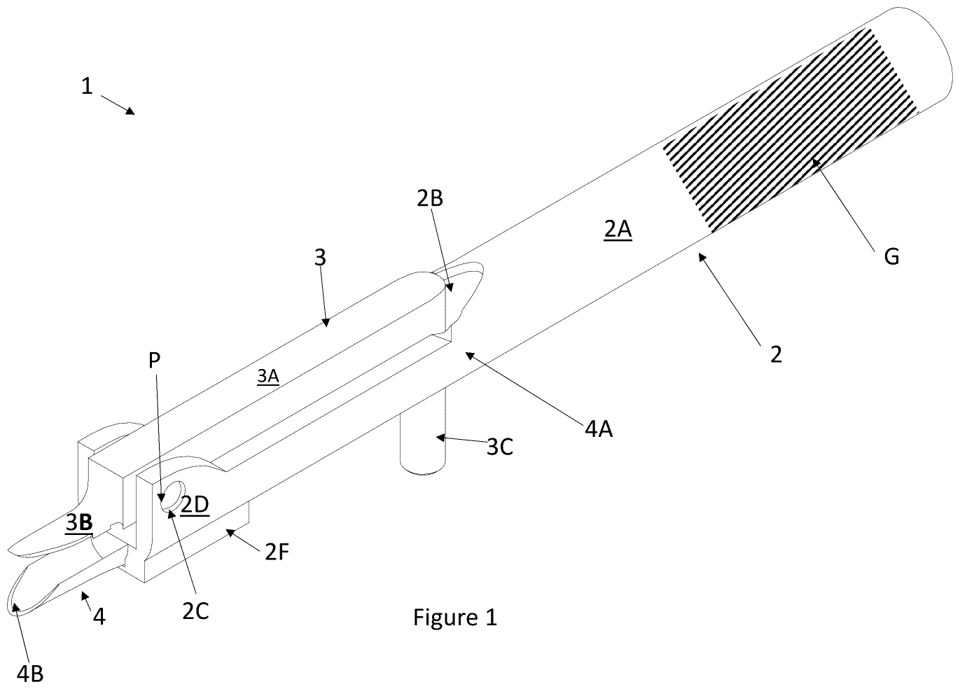

FIG. 1 shows an exemplary punch biopsy device in accordance with the disclosure in the present application. The exemplary embodiment for a punch biopsy device 1, in accordance with the present disclosure comprises an elongated body 2, a cutter actuator 3, a cutter portion 4 and a pivot element P.

The elongated body 2 comprises at least two distal ends, wherein in the first distal end 2A comprises a handle portion G. The handle portion G is configurated to include an ergonomic shape, grooves, rubber cover or any other structure that assists with the handling of the punch biopsy device 1. The second distal end comprises the cutter portion 4, cutter actuator 3 and the pivot element P. Further, the second distal end comprises an opening 2B, a set of flanges 2D, extended walls W, a flange hole 2C, a bottom surface 2E, a bottom surface hole 2H and a receiving cutter portion 2F.

As shown in FIG. 2, the cutter portion 4 comprising a cutter end 4B and an attachment element 4A is coupled to the elongated body 2 by means of the receiving cutter portion 2F in order to fix the cutter portion to the elongated body 2. The cutter portion 4 could be integrally made with the elongated body 2 to reduce costs or separate (which is disclose below). The cutter portion 4 is configured to have enough material strength to cut into a patient's skin (or the part of the body that is going to be inserted) without breaking apart by the force applied to it.

Further, as shown in FIG. 2, the opening 2B is configured to receive the cutter actuator 3. The opening 2B comprises two parallel walls W extended from the first distal end 2A, a bottom surface 2E, a bottom surface hole 2H, a set of flanges 2D extended at the distal end of the walls W, each flange 2D comprising a flange hole 2C for receiving the pivot element P.

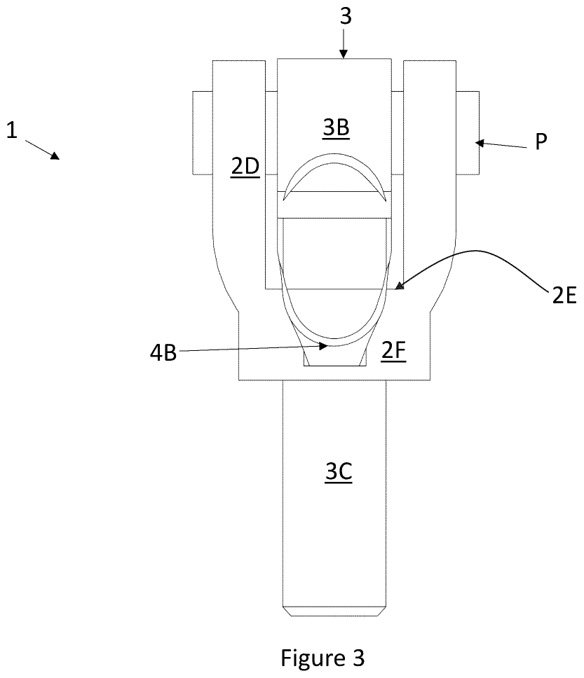

The cutter actuator 3, which is inserted in the opening 2A, as shown in FIG. 2, comprises an actuator body 3A, an upper cutter extended end 3B at the first distal end of the actuator body 3A and a thrusting element 3C at the second distal end of the actuator body 3A. The thrusting element 3C extends perpendicularly to the second distal end of the actuator body 3A and the upper cutter extended end 3B extends away, in an horizontal configuration from the actuator body 3A. Further the actuator body 3A comprises an actuator hole 3H for receiving the pivot element P. As shown in FIG. 3, the pivot element P pass through the flange 2D and the actuator hole 3A, more particularly the actuator body hole 3H, holding in place the cutter actuator 3 while providing a rotational motion of the cutter actuator 3 with respect to the pivot element P.

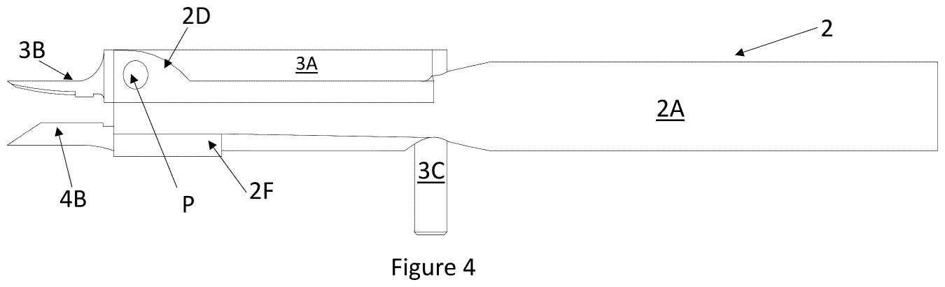

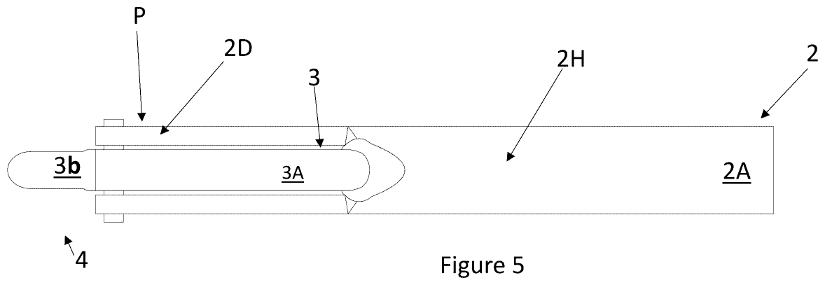

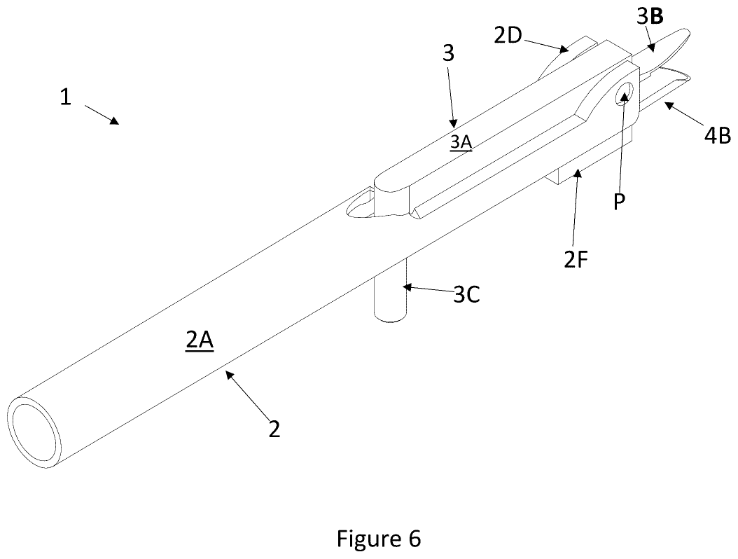

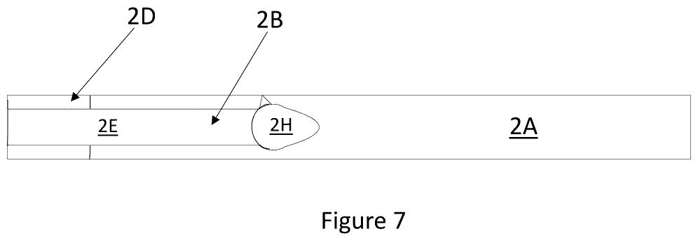

The cutter actuator 3, more particularly the thrusting element 3C is intended to pass through the bottom surface hole 2H, extending away from the bottom surface hole 2H, as shown in FIG. 4. The bottom surface hole 2H, as shown in FIG. 5, provides enough space to permit the rotational of the cutter actuator 3. FIG. 6 and FIG. 7 show how the bottom surface hole 2H is configured to receive the thrusting element 3C.

The rotational motion of the cutter actuator 3 with respect to the pivot element P is limited by the bottom surface hole 2H space and the space between the cutting portion ends, more particularly the space between the upper cutter extended end 3B and the cutter end 4B. This is due to the configuration of the actuator and the pivot element P. For example, the user, after inserting the cutting portion in a patient, pushes the thrusting element 3C, the rotational motion will close the initial distance between the upper cutter extended end 3B and the lower cutter end 4B creating a grasping tissue action at the second distal end of the punch biopsy device 1. At the same time, when the user pushes the thrusting element 3C the cutter actuator 3 moves in a rotational manner reducing the gap between the extended thrusting element 3C and the handle portion 2A. Therefore, the bottom surface hole 2H, in combination with the cutting portion ends movement, as explained above, limits the rotational movement of the cutter actuator 3.

Different shapes could be accomplished by the space between the cutting portion ends, more particularly the space between the upper cutter extended end 3B and the lower cutter end 4B. In the exemplary embodiment, as shown in FIG. 2, the space between the cutting portion ends has a semicircular shape due to the sharp cutting edges of the upper cutter extended end 3B and the lower cutter end 4B.

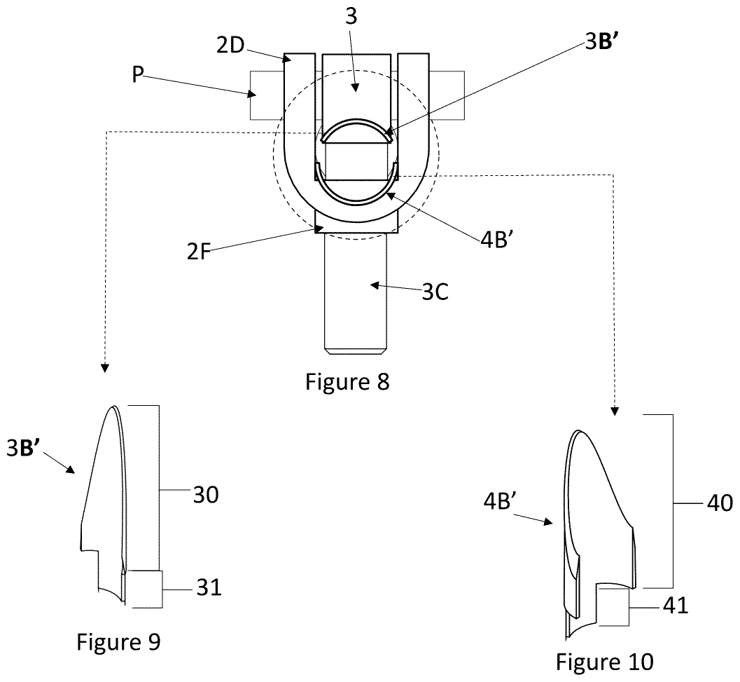

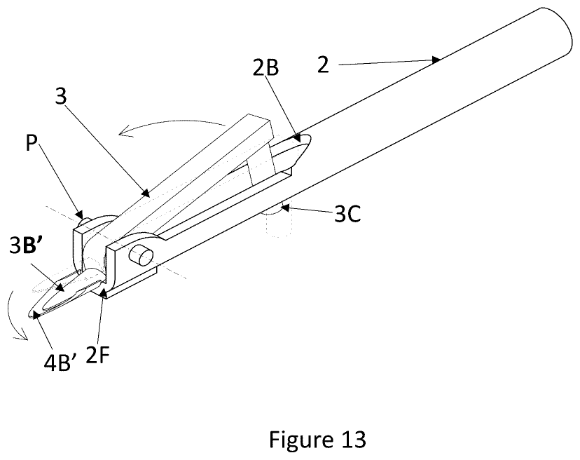

A second embodiment for the punch biopsy device 1′ is disclosed in FIG. 8 to FIG. 13. The second embodiment, as shown in FIG. 8, comprises a detachable upper cutter extended end 3B′ and a detachable lower cutter end 4B′. Both pieces are removable from the actuator body 3A and the receiving cutter portion 2F respectively for replacement. As explained above, different shapes could be accomplished by the space between the cutting portion ends, more particularly the space between the upper cutter extended end 3B′ and the lower cutter end 4B′. In the second exemplary embodiment, as shown in FIG. 8, the space between the cutting portion ends has a semicircular shape due to the sharp cutting edges of the upper cutter extended end 3B′ and the lower cutter end 4B′. The lower cutter end 4B′ is intended to receive the upper cutter extended end 3B′ in a concave configuration. The lower cutter end 4B′ receiving the upper cutter extended end 3B′ is shown in more detail in FIG. 13, which is explained below.

As shown in FIG. 9, the detachable upper cutter extended end 3B′ comprises a cutting structure 30 and upper detachable attaching element 31. While several materials or combination of materials could be selected for the detachable upper cutter extended end 3B′ it is preferable to be made with a material that withstand the pression applied by the user on the actuator body 3A in order to be inserted for capturing a tissue example without contaminating the patient. Sharp cutting edges are configured around the cutting structure 30 for a cleaner insertion of the cutting structure into patient's skin.

As shown in FIG. 10, the detachable lower cutter end 4B′ comprises a lower cutting structure 40 and a lower detachable attaching element 41. Further, while several materials or combination of materials could be selected for the detachable lower cutter end 4B′ it is preferably to be made with a material that withstand the pression applied by the user on the receiving cutter portion 2F in order to be inserted for capturing a tissue example without contaminating the patient. Sharp cutting edges are configured around the lower cutting structure 40 for a cleaner insertion of the lower cutting structure 40 into patient's skin.

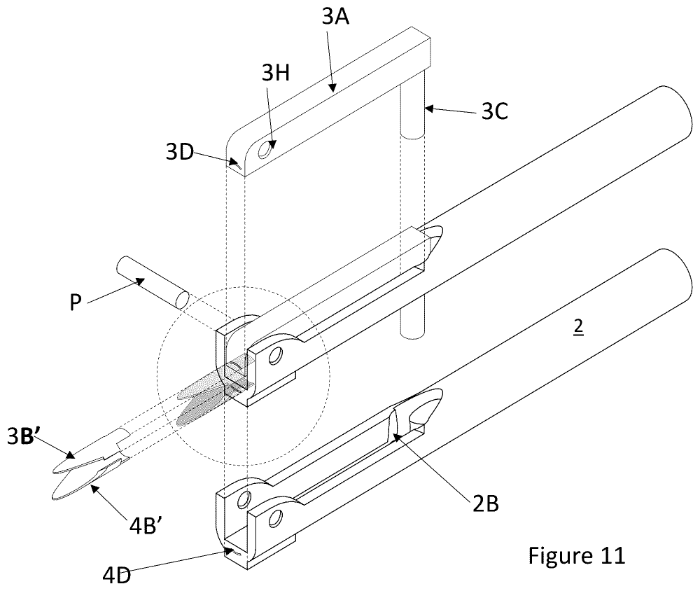

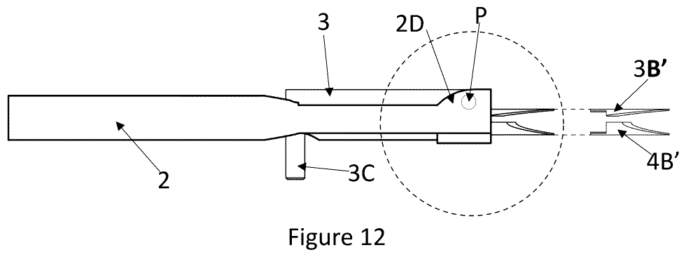

The second embodiment comprises a cutter actuator 3, which is inserted in the opening 2B, as shown in FIG. 11, comprises an actuator body 3A, a detachable upper cutter extended end 3B′ attached at the first distal end of the actuator body 3A. Actuator body 3 comprises a receiving engaging portion 3D for receiving the upper detachable attaching element 31. The receiving cutter portion 2F also comprises a receiving engaging portion 4D for receiving the lower detachable attaching element 41. FIG. 12 is a side view of the upper cutter extended end 3B′ and the lower cutter end 4B′ assembly to their respective receiving portions. The detachable upper cutter extended end 3B′ and detachable lower cutter end 4B′ are configured to stay in position while a procedure is in process, therefore the actuator receiving engaging portion 3D and the elongated body receiving engaging portion 4D may comprises additional structures to fix the cutter in position, such as magnets, removable adhesive, or others.

FIG. 13 discloses the rotational motion of the cutter actuator 3 with respect to the pivot element P is in part limited by the bottom surface hole 2H space and the space between the cutting portion ends, more particularly the space between the upper cutter extended end 3B′ and the cutter end 4B′. This is due to several factors including the configuration of the actuator, the pivot element P and shape of the cutting distal ends are considered for limiting the rotational motion. For example, the user, after inserting the cutting portion in a patient, push the thrusting element 3C toward the elongated body, the pushing action generates a rotational motion that will close or reduce the initial distance between the detachable upper cutter extended end 3B′ and the detachable lower cutter end 4B′ creating a grasping tissue action at the second distal end of the punch biopsy device 1′. The concave configuration of the detachable lower cutter end 4B′ receives the detachable upper cutter extended end 3B′. The circumference of the lower cutter end 4B′ is bigger than the detachable upper cutter extended end 3B′ as shown in FIG. 13. Due to the relation between the circumference of the two parts when the user pushes the thrusting element 3C the cutter actuator 3 moves in a rotational manner reducing the gap the detachable upper cutter extended end 3B′ and the detachable lower cutter end 4B′ resulting in a grasping action on the tissue portion between said parts.

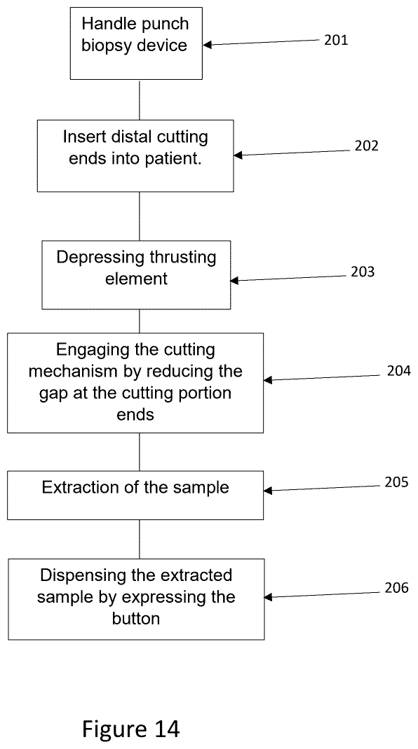

The process for using the exemplary embodiment is shown in FIG. 14. The first step 201 is for a first user to handle the punch biopsy device 1 by holding the handle portion G. Next the punch biopsy device 1 is located on top of the patient's part for acquiring a first sample. For example, on top of the patient's skin. Next step 202 is to insert the cutting portion ends into patient, more particularly the upper cutter extended end 3B and the lower cutter end 4B are inserted into patient skin. Once the cutting portion ends are inserted into patient skin the cutter actuator 3, the next step 203 is to depress or push the thrusting element. The exemplary punch biopsy device 1 is configured to achieve the next step 204. The step 204 is achieved by engaging the cutting mechanism or cutting portion ends by reducing the gap between said cutting portion ends, mainly by the rotational movement of the cutter actuator 3 with respect to the pivot element P. Reducing the gap between said cutting portion ends by continuously pressing the thrusting element 3C keeps the cutting portions ends engaged while provides a grasping action on the sample tissue. While the grasping action is completed the next step 205 for the user is to extract the sample. After extracting the sample from the patient, the next step 206 is to dispense said sample into a controlled environment, for example a sealed bag or cup, in order to complete further tests on the sample. Disengaging the reduced gap could be achieved by releasing the thrusting element 3C. Other forms of engaging and disengaging are possible such as providing a mechanism to hold the thrusting element 3C while removing the sample and releasing the thrusting element 3C while dispending the sample.

As previously disclose, the present disclosure includes an exemplary embodiment. Therefore, in some embodiments, the exemplary punch biopsy device could operate by an electronic actuator, wherein the depressing action could be achieved by electronic components in combination of the elements disclose within the scope of the present disclosure. Further, the cutting portion ends may comprise a surface that assists with the grasping action of the tissue.

1. A biopsy apparatus comprising:
an elongated body,
wherein the elongated body comprises a first distal end, a second distal end, and an opening, wherein the opening is a hollow space at the second distal end of said elongated body;
a cutter portion, wherein the cutter portion extends from the second distal end;
a cutter actuator, wherein the cutter actuator is attached to the elongated body by a pivot element and said cutter actuator is positioned in said opening, and;
wherein said cutter actuator comprises an upper cutter extended end.

2. The biopsy apparatus of claim 1, wherein the opening comprises; two parallel wall extended from the second distal end, a bottom surface, a bottom surface hole, wherein each wall comprises a distal end, and said distal end comprises a flange and wherein each flange comprises a flange hole.

3. The biopsy apparatus of claim 2, flange hole of each wall receives the pivot element.

4. The biopsy apparatus of claim 1, wherein the upper cutter extended is detachable from said cutter actuator.

5. The biopsy apparatus of claim 1, wherein the cutter portion is detachable from the second distal end.

6. The biopsy apparatus of claim 1, wherein the first distal end comprises a handle portion.

7. The biopsy apparatus of claim 6, wherein the handle portion comprises an ergonomic shape that assists with the handling of the elongated body.

8. The biopsy apparatus of claim 1, wherein the cutter actuator comprises an actuator body, the upper cutter extended end at a first distal end of the actuator body and a thrusting element at a second distal end of the actuator body.

9. The biopsy apparatus of claim 8, wherein the thrusting element passes through the bottom surface hole and extends away from the bottom surface hole.

10. The biopsy apparatus of claim 1, wherein the cutter portion comprises a sharp cutting edge.

11. The biopsy apparatus of claim 1, wherein the upper cutter extended comprises a sharp cutting edge.
